(19) United States
(12) Patent Application Publication
(10) Pub. No.: US 2016/0090360 A1
(43) Pub. Date: Mar. 31, 2016

(54) SYNTHESIS OF BACE INHIBITORS

(71) Applicant: HOFFMANN-LA ROCHE INC., Nutley, NJ (US)

(72) Inventors: Alec Fettes, Basel (CH); Hans-Peter Marty, Basel (CH); Michelangelo Scalone, Birsfelden (CH)

(21) Appl. No.: 14/785,659

(22) PCT Filed: Apr. 23, 2014
(86) PCT No.: PCT/EP2014/058172

§ 371 Date: Oct. 20, 2015

(30) Foreign Application Priority Data
Apr. 26, 2013 (EP) 13165634.0

(51) Int. Cl. C07D 213/84; C07D 213/79; C07D 413/12; C07D 213/803

(52) U.S. Cl. CPC C07D 213/84; C07D 213/803; C07D 213/79; C07D 413/12

(57) ABSTRACT

The present invention provides a one step carbonylation of a compound of formula II in the presence of water to afford a compound of formula (I), useful in processes to manufacture BACE inhibitors.

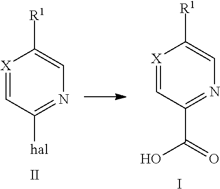

### FIELD OF THE INVENTION

The present invention provides processes to manufacture BACE inhibitors.

### BACKGROUND OF THE INVENTION

WO 2011/069934¹, WO2011070029² describe certain BACE inhibitors. WO 2004/071440³ relates to thiazolyl-based compounds useful for treating p38 kinase-associated conditions and it describes a palladium-catalyzed esterification of an aryl halide. US 2009/209755⁴ relates to fused aminohydrothiazines useful for treating BACE associated condition and it describes the hydrolysis of aryl esters to the corresponding acid. Colquhoun et al⁵. describes a carbonylation of an aryl halide directly in the presence of water to the corresponding carboxylic acid. This reaction type is usually performed in water free medium (Pri-Bar et al.⁶) or in complex ionic liquids as solvents (Mizushima et al.⁷).

### DETAILED DESCRIPTION OF THE INVENTION

The present invention provides processes to manufacture BACE inhibitors as well as intermediates.

Present invention relates to a one step carbonylation of a compound of formula II in the presence of water to afford a compound of formula I,

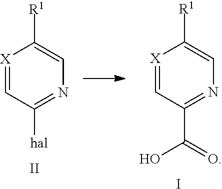

It was surprisingly found that the direct conversion (one step reaction) of a compound of formula II to a compound of formula I in the presence of water proceeded with a high chemoselectivity, can be performed under mild conditions and in the presence of small amounts of a catalyst.

### DEFINITIONS

The following definitions of the general terms used in the present description apply irrespectively of whether the terms in question appear alone or in combination with other groups.

The term “room temperature” refers to 18-30° C., in particular 20-25° C., more particular to 20° C.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl, 1,2-dimethyl-propyl and the like. Particular “C₁₋₆-alkyl” groups are “C₁₋₃-alkyl”. Specific groups are methyl and ethyl. Most specific is methyl.

The term “halogen-C₁₋₆-alkyl”, alone or in combination with other groups, refers to C₁₋₆-alkyl as defined herein, which is substituted by one or multiple halogen, particularly 1-5 halogen, more particularly 1-3 halogen. Particular halogen is fluoro. Particular “halogen-C₁₋₆-alkyl” is fluoro-C₁₋₆-alkyl and a particular “halogen-C₁₋₃-alkyl” is fluoro-C₁₋₃-alkyl. Examples are trifluoromethyl, difluoromethyl, fluoromethyl and the like. A specific group is —CHF₂.

The terms “halo”, “halogen” and “halide”, which may be used interchangeably, refer to a substituent fluoro, chloro, bromo, or iodo. A specific “halogen” is fluoro.

The term “cyano” refers to —CN.

The term “C₁₋₆-alkoxy”, alone or in combination with other groups, stands for an —O—C₁₋₆-alkyl radical which may be linear or branched, with single or multiple branching, wherein the alkyl group in general comprises 1 to 6 carbon atoms, for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular “C₁₋₆-alkoxy” groups have 1 to 4 carbon atoms. A specific group is methoxy.

The term “halogen-C₁₋₆-alkoxy”, alone or in combination with other groups, refers to C₁₋₆-alkoxy as defined herein, which is substituted by one or multiple halogens, in particular fluoro. Particular “halogen-C₁₋₆-alkoxy” groups are fluoro-C₁₋₆-alkoxy. A specific “halogen-C₁₋₆-alkoxy” group is trifluoromethoxy.

The term “as defined herein” and “as described herein” when referring to a variable incorporates by reference the broad definition of the variable as well as particularly, more particularly and most particularly definitions, if any.

The term “chemoselectivity” is meant qualitatively as the preferential outcome of the desired reaction over a set of other plausible reactions.

The term “aromatic” denotes the conventional idea of aromaticity as defined in the literature, in particular in IUPAC—Compendium of Chemical Terminology⁸.

Whenever a chiral carbon is present in a chemical structure, it is intended that all stereoisomers associated with that chiral carbon are encompassed by the structure as pure stereoisomers as well as mixtures thereof.

“Solution” as used herein is meant to encompass liquids wherein a reagent or reactant is present in a solvent in dissolved form (as a solute) or is present in particulate, undissolved form, or both. Thus, in a “solution”, it is contemplated that the solute may not be entirely dissolved therein and solid solute may be present in dispersion or slurry form. Accordingly, a “solution” of a particular reagent or reactant is meant to encompasses slurries and dispersions, as well as solutions, of such reagents or reactants. “Solution” and “Slurry” may be used interchangeable herein.

“Solvent” as used herein is meant to encompass liquids that fully dissolve a reagent or reactant exposed to the solvent, as well as liquids which only partially dissolve the reagent or reactant or which act as dispersants for the reagent or reactant. Thus, when a particular reaction is carried out in a “solvent”, it is contemplated that some or all of the reagents or reactants present may not be in dissolved form.

The term “pharmaceutically acceptable salts” denotes salts which are not biologically or otherwise undesirable. Pharmaceutically acceptable salts include both acid and base addition salts. The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid. The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

A certain embodiment of the invention relates to a one step carbonylation of a compound of formula II in the presence of water to afford a compound of formula I,

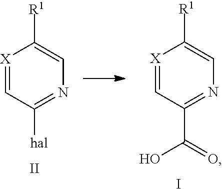

wherein

### hal is Cl or Br;

### X is —C—R² or N;

R¹ is selected from the group consisting of
i) halo-C₁₋₆alkyl,
ii) C₁₋₆alkyl,
iii) halo-C₁₋₆alkoxy,
iv) C₁₋₆alkoxy, and
v) cyano,
   R² is selected from the group consisting of
i) C₁₋₆alkyl, and
ii) hydrogen.

A certain embodiment of the invention relates to the carbonylation as described herein, wherein R¹ is cyano.

A certain embodiment of the invention relates to the carbonylation as described herein, wherein R¹ is C₁₋₆alkoxy.

A certain embodiment of the invention relates to the carbonylation as described herein, wherein R¹ is methoxy.

A certain embodiment of the invention relates to the carbonylation as described herein, wherein X is —C—R².

A certain embodiment of the invention relates to the carbonylation as described herein, wherein R² is hydrogen.

A certain embodiment of the invention relates to the carbonylation as described herein, wherein R² is C₁₋₆alkyl.

A certain embodiment of the invention relates to the carbonylation as described herein, wherein R² is methyl.

A certain embodiment of the invention relates to the carbonylation as described herein, wherein X is N.

A certain embodiment of the invention relates to the carbonylation as described herein, wherein hal is Cl.

A certain embodiment of the invention relates to the carbonylation as described herein, wherein hal is Br.

A certain embodiment of the invention relates to the carbonylation as described herein, wherein hal is Cl; X is —CH and R¹ is cyano.

A certain embodiment of the invention relates to the carbonylation as described herein, wherein hal is Br; X is —C—CH₃ and R¹ is cyano.

A certain embodiment of the invention relates to the carbonylation as described herein, wherein hal is Br; X is N and R¹ is methoxy.

A certain embodiment of the invention relates to the carbonylation as described herein, using a palladium catalyst.

A certain embodiment of the invention relates to the carbonylation as described herein, using PdCl₂(dppp) as catalyst.

A certain embodiment of the invention relates to the carbonylation as described herein, performed under a CO pressure (p_{CO}) of 1≦p_{CO}≦200 bar.

A certain embodiment of the invention relates to the carbonylation as described herein, performed under a CO pressure (p_{CO}) of 15≦p_{CO}≦100 bar.

A certain embodiment of the invention relates to the carbonylation as described herein, performed under a CO pressure (p_{CO}) of 15≦p_{CO}≦40 bar.

A certain embodiment of the invention relates to the carbonylation as described herein, performed under a CO pressure (p_{CO}) of 15 bar.

A certain embodiment of the invention relates to the carbonylation as described herein, performed at a temperature (t) of RT≦t≦150° C.

A certain embodiment of the invention relates to the carbonylation as described herein, performed at a temperature (t) of 40≦t≦100° C.

A certain embodiment of the invention relates to the carbonylation as described herein, performed at a temperature (t) of 50≦t≦80° C.

A certain embodiment of the invention relates to the carbonylation as described herein, performed at a temperature (t) of 60≦t≦70° C.

A certain embodiment of the invention relates to the carbonylation as described herein, performed at a temperature (t) of 60° C.

A certain embodiment of the invention relates to the carbonylation as described herein, using a mixture of the following solvents with water: dioxane, acetonitrile, acetone, methyl ethylketone, tert-butanol, DMF, THF, 2-methyl-THF, dimethoxyethane or DMSO.

A certain embodiment of the invention relates to the carbonylation as described herein, using a mixture of water and dioxane.

A certain embodiment of the invention relates to the carbonylation as described herein, using a mixture of water and acetonitrile.

A certain embodiment of the invention relates to the carbonylation as described herein, using a mixture of water and acetone.

A certain embodiment of the invention relates to the carbonylation as described herein, using a mixture of water and methyl ethylketone.

A certain embodiment of the invention relates to the carbonylation as described herein, using a mixture of water and tert-butanol.

A certain embodiment of the invention relates to the carbonylation as described herein, using a mixture of water and DMF.

A certain embodiment of the invention relates to the carbonylation as described herein, using a mixture of water and THF.

A certain embodiment of the invention relates to the carbonylation as described herein, using a mixture of water and 2-methyl-THF.

A certain embodiment of the invention relates to the carbonylation as described herein, using a mixture of water and dimethoxyethane.

A certain embodiment of the invention relates to the carbonylation as described herein, using a mixture of water and DMSO.

A certain embodiment of the invention relates to the carbonylation as described herein, using a mixture of water and a solvent as described herein in a ratio (vol/vol) of 1:0.

A certain embodiment of the invention relates to the carbonylation as described herein, using a mixture of water and a solvent as described herein in a ratio (vol/vol) of 1:1.

A certain embodiment of the invention relates to the carbonylation as described herein, using a mixture of water and a solvent as described herein in a ratio (vol/vol) of 2:1.

A certain embodiment of the invention relates to the carbonylation as described herein, using a mixture of water and a solvent as described herein in a ratio (vol/vol) of 1:2.

A certain embodiment of the invention relates to the carbonylation as described herein, using a mixture of water and a solvent as described herein in a ratio (vol/vol) of 1:4.

A certain embodiment of the invention relates to the carbonylation as described herein, using a mixture of water and tert-butanol in a ratio (vol/vol) of 1:1.

A certain embodiment of the invention relates to the carbonylation as described herein, using a mixture of water and tert-butanol in a ratio (vol/vol) of 2:1.

A certain embodiment of the invention relates to the carbonylation as described herein, using a mixture of water and tert-butanol in a ratio (vol/vol) of 1:2.

A certain embodiment of the invention relates to the carbonylation as described herein, using a mixture of water and tert-butanol in a ratio (vol/vol) of 1:4.

A certain embodiment of the invention relates to the carbonylation as described herein, using one of the following bases: Et₃N, NaOAc, KOAc, NH₄OAc, NaHCO₃, KHCO₃, NaOH, Na₂CO₃, K₂CO₃, (i-Pr)₂NEt, Bu₃N, Cy₂NH, K₂HPO₄ or Na₂SO₄.

A certain embodiment of the invention relates to the carbonylation as described herein, using Et₃N as base.

A certain embodiment of the invention relates to the carbonylation as described herein, using NaOAc as base.

A certain embodiment of the invention relates to the carbonylation as described herein, using KOAc as base.

A certain embodiment of the invention relates to the carbonylation as described herein, using NH₄OAc as base.

A certain embodiment of the invention relates to the carbonylation as described herein, using NaHCO₃ as base.

A certain embodiment of the invention relates to the carbonylation as described herein, using KHCO₃ as base.

A certain embodiment of the invention relates to the carbonylation as described herein, using NaOH as base.

A certain embodiment of the invention relates to the carbonylation as described herein, using Na₂CO₃ as base.

A certain embodiment of the invention relates to the carbonylation as described herein, using K₂CO₃ as base.

A certain embodiment of the invention relates to the carbonylation as described herein, using (i-Pr)₂NEt as base.

A certain embodiment of the invention relates to the carbonylation as described herein, using Bu₃N as base.

A certain embodiment of the invention relates to the carbonylation as described herein, using Cy₂NH as base.

A certain embodiment of the invention relates to the carbonylation as described herein, using K₂HPO₄ as base.

A certain embodiment of the invention relates to the carbonylation as described herein, using Na₂SO₄ as base.

A certain embodiment of the invention relates to the carbonylation as described herein, using no base.

A certain embodiment of the invention relates to the carbonylation as described herein to synthesise a compound of formula I, further comprising a compound of formula I reacting with a compound of formula V to a compound of formula VI.

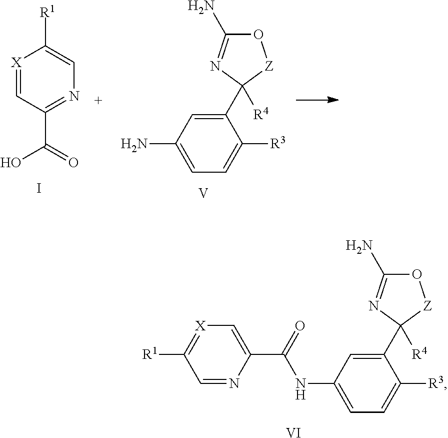

wherein R¹ and X have the meaning as described in any of claims 1-14, and
Z is —C(R⁵,R⁶)—C(R⁷,R⁸)—;
R³ is selected from the group consisting of
i) hydrogen, and
ii) halogen,
   R⁴ is selected from the group consisting of
i) hydrogen,
ii) halo-C₁₋₆alkyl, and
iii) halogen,
   R⁵, R⁶, R⁷ and R⁸ are each independently selected from the group consisting of
i) hydrogen,
ii) halo-C₁₋₆alkyl, and
iii) halogen;
   or a pharmaceutically acceptable salt thereof.

A certain embodiment of the invention relates to the process as described herein, wherein R³ is F.

A certain embodiment of the invention relates to the process as described herein, wherein R¹ is cyano.

A certain embodiment of the invention relates to the process as described herein, wherein X is —CH.

A certain embodiment of the invention relates to the process as described herein, wherein R⁴ is C₁₋₆alkyl.

A certain embodiment of the invention relates to the process as described herein, wherein R⁴ is methyl.

A certain embodiment of the invention relates to the process as described herein, wherein R⁵ and R⁶ are both hydrogen.

A certain embodiment of the invention relates to the process as described herein, wherein R⁵ and R⁶ are both halogen.

A certain embodiment of the invention relates to the process as described herein, wherein R⁵ and R⁶ are both fluoro.

A certain embodiment of the invention relates to the process as described herein, wherein R⁷ and R⁸ are both hydrogen.

A certain embodiment of the invention relates to the process as described herein, wherein R⁷ and R⁸ are both halogen.

A certain embodiment of the invention relates to the process as described herein, wherein R⁷ and R⁸ are both fluoro.

A certain embodiment of the invention relates to the process as described herein, wherein R¹ is methoxy, R³ is F, R⁴ is methyl, X is —CH, R⁵ and R⁶ are both hydrogen and R⁷ and R⁸ are both fluoro.

A certain embodiment of the invention relates to the process as described herein, wherein R¹ is methoxy, R³ is F, R⁴ is methyl, X is —CH, R⁵ and R⁶ are both hydrogen and R⁷ and R⁸ are both hydrogen.

A certain embodiment of the invention relates to the process as described herein, wherein R¹ is cyano, R³ is F, R⁴ is methyl, X is —CH, R⁵ and R⁶ are both hydrogen and R⁷ and R⁸ are both fluoro.

A certain embodiment of the invention relates to the process as described herein, wherein R¹ is cyano, R³ is F, R⁴ is methyl, X is —CH, R⁵ and R⁶ are both hydrogen and R⁷ and R⁸ are both hydrogen.

A certain embodiment of the invention relates to the process as described herein, wherein R¹ is methoxy, R³ is F, R⁴ is —CHF₂, X is —CH, R⁵ and R⁶ are both hydrogen and R⁷ and R⁸ are both fluoro.

A certain embodiment of the invention relates to the process as described herein, wherein R¹ is methoxy, R³ is F, R⁴ is —CHF₂, X is —CH, R⁵ and R⁶ are both hydrogen and R⁷ and R⁸ are both hydrogen.

A certain embodiment of the invention relates to the process as described herein, wherein R¹ is cyano, R³ is F, R⁴ is —CHF₂, X is —CH, R⁵ and R⁶ are both hydrogen and R⁷ and R⁸ are both fluoro.

A certain embodiment of the invention relates to the process as described herein, wherein R¹ is cyano, R³ is F, R⁴ is —CHF₂, X is —CH, R⁵ and R⁶ are both hydrogen and R⁷ and R⁸ are both hydrogen.

A certain embodiment of the invention relates to the process as described herein, wherein R¹ is methoxy, R³ is F, R⁴ is —CH₂F, X is —CH, R⁵ and R⁶ are both hydrogen and R⁷ and R⁸ are both fluoro.

A certain embodiment of the invention relates to the process as described herein, wherein R¹ is methoxy, R³ is F, R⁴ is —CH₂F, X is —CH, R⁵ and R⁶ are both hydrogen and R⁷ and R⁸ are both hydrogen.

A certain embodiment of the invention relates to the process as described herein, wherein R¹ is cyano, R³ is F, R⁴ is —CH₂F, X is —CH, R⁵ and R⁶ are both hydrogen and R⁷ and R⁸ are both fluoro.

A certain embodiment of the invention relates to the process as described herein, wherein R¹ is cyano, R³ is F, R⁴ is —CH₂F, X is —CH, R⁵ and R⁶ are both hydrogen and R⁷ and R⁸ are both hydrogen.

A certain embodiment of the invention relates to a compound of formula I, whenever synthesized via a carbonylation as described herein.

A certain embodiment of the invention relates to a compound of formula VI, whenever synthesized via a process as described herein.

A certain embodiment of the invention relates to a compound of formula VI, synthesized by a process as described herein, for the use as therapeutically active substance for the therapeutic and/or prophylactic treatment of diseases and disorders characterized by elevated β-amyloid levels and/or β-amyloid oligomers and/or β-amyloid plaques and further deposits, particularly Alzheimer's disease.

A pharmaceutical composition comprising a compound of formula VI, synthesized by a process as described herein, and a pharmaceutically acceptable carrier and/or a pharmaceutically acceptable auxiliary substance.

### DETAILED DESCRIPTION OF THE INVENTION

### Experimental Part

The following experiments are provided for illustration of the invention. They should not be considered as limiting the scope of the invention, but merely as being representative thereof.

### Abbreviations

PdCl₂(dppp): Dichloro[1,1′-bis(diphenylphosphino)propane]palladium(II), CAS No. 59831-02-6
P(3,5-tBu)₃: Tris-(3,5-di-tert-butyl-phenyl)-phosphane
dppb: 1,1′-bis(diphenylphosphino)butane
dppf: 1,2-bis(diphenylphosphino)ferrocene
DiPrPF: 1,2-bis(di-isopropylphosphino)ferrocene
BIPHEP: 2,2′-bis(diphenylphosphino)1,1′-biphenyl
CO: carbon monoxide
S/C: substrate-to-catalyst molar ratio
3-CN-Py: 3-cyanopyridine
2-Cl,5-CN-Py: 2-Chloro-5-cyanopyridine
3-CN-Py-2-CO2H: 5-cyano-pyridine-2-carboxylic acid
3-CN-Py-2-CO2Me: 5-cyano-pyridine-2-carboxylic acid methyl ester
THF: tetrahydrofuran
DMSO: dimethyl sulfoxide

### DMF: N,N-Dimethylformamid

2-methyl-THF: 2-Methyltetrahydrofuran
Et₃N: triethylamine
NaOAc: sodium acetate
KOAc: potassium acetate
NH₄OAc: ammonium acetate
NaHCO₃: sodium bicarbonate
KHCO₃: potassium bicarbonate
NaOH: sodium hydroxide
Na₂CO₃: sodium carbonate
K₂CO₃: potassium carbonate
(i-Pr)₂NEt: diisopropylethylamine (Hüning's base)

### Bu₃N: N-tributylamine

Cy₂NH: Bis(dicyclo-hexyl-amine
K₂HPO₄: Potassium monohydrogen phosphate
Na₂SO₄: sodium sulfate
t-BuOH: tert-butanol
m.p.: melting point (uncorrected)
a %: area % measured in the analytic method indicated (GC or HPLC)
n.d.: not determined

### Example 1

### 5-Cyano-pyridine-2-carboxylic acid

A 2 L stirred autoclave was charged under argon with PdCl₂(dppp) (2.13 g, 3.61 mmol), 6-chloro-nicotinonitrile (100 g, 0.722 mol), tert-butanol (800 ml), deionized water (200 ml) and triethylamine (250 ml, 1.8 mol). The reaction vessel was closed, purged three times with carbon monoxide (10 bar) and finally charged with carbon monoxide to 15 bar. The mixture was stirred vigorously at 60° C. under constant pressure for 10 h; after this time no more carbon monoxide absorption was observed. The reaction mixture was concentrated on a rotary evaporator such that the volatile organic components were removed. The resulting aqueous phase was filtered, extracted with dichloromethane and treated with active charcoal. After filtration, the pH of the solution was reduced under stirring at 60° C. to ca. 0.7 by dropwise addition of hydrochloric acid. The resulting suspension was stirred at room temperature over night and then filtered. The filter cake was rinsed with water and dried in vacuo to constant weight to afford 5-cyano-pyridine-2-carboxylic acid (98.95 g) as a white solid, MS: m/z=104 [M−CO₂], m.p.: 207° C. (dec).

### Example 2

### 5-Cyano-pyridine-2-carboxylic acid

A 35 ml autoclave equipped with a magnetic stirring bar was charged under argon with PdCl₂(dppp) (8.85 mg, 0.015 mmol), 6-chloro-nicotinonitrile (416 mg, 3.0 mmol), tert-butanol (2 ml), deionized water (2 ml) and triethylamine (1.04 ml, 7.51 mmol). The reaction vessel was closed, purged three times with carbon monoxide (40 bar) and finally charged with carbon monoxide to 40 bar. The mixture was stirred vigorously at 60° C. After 6 h the autoclave was opened and the reaction mixture was analyzed: 20 μl of reaction mixture were diluted in a mixture consisting of 0.8 ml of acetonitrile, 0.2 ml of water and 5 drops of 1 M HCl and analyzed by HPLC. Only 0.8 a % of 6-chloro-nicotinonitrile were present, the mail peaks being 5-cyano-pyridine-2-carboxylic acid (95.8 a %) and 3-CN-py (1.1 a %).

### Examples 3a-f

A series of palladium complexes was tested as (pre)catalysts using the same procedure described in Example 2. The results are included in the following table:

| | 2-Cl,5-CN-Py | 3-CN-Py-2-CO₂H | 3-CN-Py | Sel. | |
|---|---|---|---|---|---|
| Ex. | [a %] | [a %] | [a %] | % | Catalyst |
| 3a | 80.9 | 9.7 | 2.6 | 51 | PdCl₂(PPh₃)₂ ^{a)} |
| 3b | 45.7 | 21.6 | 11.7 | 40 | PdCl₂(P(3,5-tBu)₃)₂ ^{b)} |
| 3c | 15.6 | 78.5 | 2.9 | 93 | PdCl₂(dppb) ^{a)} |
| 3d | 60.3 | 25.0 | 6.4 | 63 | PdCl₂dppf ^{a)} |
| 3e | 0.4 | 96.7 | 0.7 | 97 | PdCl₂(DiPrPF) ^{a)} |
| 3f | 51.2 | 41.2 | 2.8 | 84 | PdCl₂(BIPHEP) ^{b,c)} |

^{a)} Commercially available.
^{b)} Prepared from PdCl₂(acetonitrile)₂ and P(3,5-tBu)₃
^{c)}a % values are obtained by HPLC analysis.

### Example 4

### 5-Cyano-pyridine-2-carboxylic acid

A 185 ml stirred autoclave was charged under argon with PdCl₂(dppp) (213 mg, 0.361 mmol), 6-chloro-nicotinonitrile (10 g, 72.2 mmol), dioxane (50 ml), deionized water (50 ml) and sodium bicarbonate (15.2 g, 0.18 mol, 2.5 molar equivalents). The reaction vessel was closed, purged three times with carbon monoxide (15 bar) and finally charged with carbon monoxide to 60 bar. The mixture was stirred vigorously at 60° C. under constant pressure for 22 h; after this time no more carbon monoxide absorption was observed. The reaction mixture was transferred to a round-bottomed flask with aid of water and, after having removed the organic volatile components on a rotary evaporator, the reaction mixture was worked-up as reported in Example 1. Crystallization afforded 5-cyano-pyridine-2-carboxylic acid (9.55 g) as a white solid with 99.6 a % purity by HPLC, MS: m/z=104 [M−CO₂].

### Example 4a

### 5-Cyano-pyridine-2-carboxylic acid

A 185 ml stirred autoclave was charged under argon with PdCl₂(dppp) (213 mg, 0.361 mmol), 6-chloro-nicotinonitrile (10.1 g, 72.2 mmol), tert-butanol (80 ml), deionized water (20 ml) and triethylamine (18.3 g, 0.18 mol, 2.5 molar equivalents). The reaction vessel was closed, purged four times with carbon monoxide (7 bar) and finally charged with carbon monoxide to 15 bar. The mixture was stirred vigorously at 60° C. under constant pressure for 10 h; after this time no more carbon monoxide absorption was observed.

The reaction mixture was concentrated on a rotary evaporator under simultaneous addition of water in order to remove the volatile organic components. The resulting aqueous phase was extracted with dichloromethane and treated with active charcoal. After filtration, the pH of the solution was reduced under stirring at 60° C. to ca. 0.7 by dropwise addition of hydrochloric acid. The resulting suspension was stirred at room temperature over night and then filtered. The filter cake was rinsed with water and dried in vacuo to constant weight to afford 5-cyano-pyridine-2-carboxylic acid (9.85 g) as a white solid, MS: m/z=104 [M−CO₂], m.p.: 207° C. (dec).

### Example 5a-h

### 5-Cyano-pyridine-2-carboxylic acid

A series of reaction conditions was tested using the same procedure described in Example 4. The results are included in the following table.

| | | | | Solvent/ | | | | Isol. |
|---|---|---|---|---|---|---|---|---|
| | P | T | Base/molar | vol/vol | 2-Cl,5-CN-Py | 3-CN-Py-2-CO₂H | 3-CN-Py | Yield |
| Ex. | bar | ° C. | equivalents | ratio | [a %] | [a %] | [a %] | (%) |
| 5a | 60 | 60 | Na₂CO₃ 1.25 | Water 1 | 0.2 | 93.5 | 1.6 | 89 |
| | | | | Dioxane 1 | | | | |
| 5b | 60 | 60 | NaOAc 2.5 | Water 1 | 3.6 | 83.5 | 7.9 | n.d. |
| | | | | Acetone 1 | | | | |
| 5c | 40 | 60 | Et₃N 2.5 | Water 1 | 0.7 | 95.9 | 1.0 | 89 |
| | | | | t-BuOH 1 | | | | |
| 5d | 15 | 60 | Et₃N 2.5 | Water 1 | <0.1 | 95.4 | 1.9 | 92 |
| | | | | t-BuOH 4 | | | | |
| 5e | 70 | 100 | Et₃N 2.2 | Water 1 | 0.4 | 53.8 | 25 | n.d. |
| | | | | THF 4 | | | | |
| 5f | 35 | 80 | Et₃N 2.5 | Water 1 | <0.1 | 93.5 | 4.9 | n.d. |
| | | | | CH₃CN 4 | | | | |
| 5g | 35 | 80 | Et₃N 2.5 | Water 1 | <0.1 | 91.5 | 1.5 | n.d. |
| | | | | CH₃CN 4 | | | | |
| | | | | MeOH 1 | | | | |
| 5h | 40 | 50 | Et₃N 2.5 | Water 1 | 10.5 | 86.6 | 0.5 | n.d. |
| | | | | t-BuOH 1 | | | | |

Example no. 5c: reaction time 5 h; example no. 5d and 5h: reaction time 16 h; example no. 5f: reaction time 12 h.

Examples no. 5e and 5f: 5 g of 2-Cl,5-CN-Py, S/C 330.

Example no. 5g: crude mixture contains 3.7 a % of the methyl ester 3-CN-Py-2-CO₂Me. a % values are obtained by HPLC analysis.

### Example 6

### 5-Cyano-4-methyl-pyridine-2-carboxylic acid

A 185 ml stirred autoclave was charged under argon with PdCl₂(dppp) (479 mg, 0.796 mmol), 6-bromo-4-methylnicotinonitrile (7.84 g, 39.8 mmol), tert-butanol (40 ml), deionized water (40 ml) and triethylamine (10.1 g, 99.5 mmol, 2.5 molar equivalents). The reaction vessel was closed, purged three times with carbon monoxide (15 bar) and finally charged with carbon monoxide to 40 bar. The mixture was stirred vigorously at 60° C. under constant pressure for 18 h; after this time no more carbon monoxide absorption was observed.

The reaction mixture was concentrated on a rotary evaporator under simultaneous addition of water in order to remove the volatile organic components. The resulting aqueous phase was extracted twice with dichloromethane and treated with active charcoal. After filtration, the pH of the solution was reduced under stirring to ca. 1 by dropwise addition of hydrochloric acid. The resulting suspension was stored at 4° C. over night and then filtered. The filter cake was rinsed with water and dried in vacuo to constant weight to afford 4-methyl-5-cyano-pyridine-2-carboxylic acid (6.45 g) as a light yellow solid, MS: m/z=163.2 [M+H].

### Example 7

### 5-Methoxypyrazine-2-carboxylic acid

A 185 ml stirred autoclave was charged under argon with PdCl₂(dppp) (508 mg, 0.844 mmol), 2-bromo-5-methoxypyrazine (8.40 g, 42.2 mmol), tert-butanol (35 ml), deionized water (45 ml) and triethylamine (10.7 g, 0.106 mol, 2.5 molar equivalents). The reaction vessel was closed, purged three times with carbon monoxide (15 bar) and finally charged with carbon monoxide to 10 bar. The mixture was stirred vigorously at 60° C. under constant pressure for 48 h; after this time no more carbon monoxide absorption was observed.

The reaction mixture was concentrated on a rotary evaporator under simultaneous addition of water in order to remove the volatile organic components. The resulting aqueous phase was extracted twice with dichloromethane. After filtration, the pH of the solution was reduced under stirring to ca. 1 by dropwise addition of hydrochloric acid. The resulting suspension was stored at 4° C. over night and then filtered. The filter cake was rinsed with water and dried in vacuo to constant weight to afford 5-methoxypyrazine-2-carboxylic acid (5.9 g) as a white solid, MS: m/z=155.2 [M+H].

### Example 8

### 5-Cyano-pyridine-2-carboxylic acid [3-((S)-2-amino-4-methyl-5,6-dihydro-4H-[1,3]oxazin-4-yl)-4-fluoro-phenyl]-amide

Condensation of 5-cyano-pyridine-2-carboxylic acid (example 1) with [(S)-4-(5-amino-2-fluoro-phenyl)-4-methyl-5,6-dihydro-4H-[1,3]oxazin-2-yl]-carbamic acid tert-butyl ester (see ²).

### Example 9

### 5-Methoxy-pyridine-2-carboxylic acid [3-((S)-2-amino-4-methyl-5,6-dihydro-4H-[1,3]oxazin-4-yl)-4-fluoro-phenyl]-amide

Condensation of 5-methoxy-pyridine-2-carboxylic acid with [(S)-4-(5-amino-2-fluoro-phenyl)-4-methyl-5,6-dihydro-4H-[1,3]oxazin-2-yl]-carbamic acid tert-butyl ester (see ²).

### Example 10

### 5-Cyano-pyridine-2-carboxylic acid [3-((R)-2-amino-5,5-difluoro-4-methyl-5,6-dihydro-4H-[1,3]oxazin-4-yl)-4-fluoro-phenyl]-amide

Condensation of 5-cyano-pyridine-2-carboxylic acid (example 1) with (R)-4-(5-amino-2-fluoro-phenyl)-5,5-difluoro-4-methyl-5,6-dihydro-4H-[1,3]oxazin-2-ylamine (see ¹).
¹ WO 2011/069934
² WO2011/070029
³ WO 2004/071440
⁴ US 2009/209755
⁵ H. M. Colquhoun, D. J. Thompson, M. V. Twigg, “Carbonylation, Direct Synthesis of Carbonyl Compounds”, Plenum Press, New York and London, 1991, page 97-99
⁶ I. Pri-Bar, O. Buchman, J. Org. Chem. 1988, 53, 624
⁷ E. Mizushima, T. Hayashi and M. Tanaka, Topics in Catalysis Vol. 29, Nos. 3-4, June 2004, page 163
⁸ Compendium of Chemical Terminology, 2nd, A. D. McNaught & A. Wilkinson (Eds). Blackwell Scientific Publications, Oxford (1997)

1. A one step carbonylation of a compound of formula II in the presence of water to afford a compound of formula I, 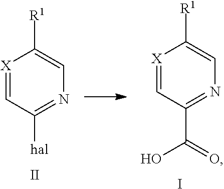
wherein
hal is Cl or Br;
X is —C—R² or N;
R¹ is selected from the group consisting of
i) halo-C₁₋₆alkyl,
ii) C₁₋₆alkyl,
iii) halo-C₁₋₆alkoxy,
iv) C₁₋₆alkoxy, and
v) cyano,
R² is selected from the group consisting of
i) C₁₋₆alkyl, and
ii) hydrogen.

2. The carbonylation according to claim 1, wherein R¹ is cyano.

3. The carbonylation according to claim 1, wherein R¹ is C₁₋₆alkoxy.

4. The carbonylation according to claim 3, wherein R¹ is methoxy.

5. The carbonylation according to claim 1, wherein X is —C—R².

6. The carbonylation according to claim 1, wherein R² is hydrogen.

7. The carbonylation according to claim 1, wherein R² is C₁₋₆alkyl.

8. The carbonylation according to claim 7, wherein R² is methyl.

9. The carbonylation according to claim 1, wherein X is N.

10. The carbonylation according to claim 1, wherein hal is Cl.

11. The carbonylation according to claim 1, wherein hal is Br.

12. The carbonylation according to claim 1, wherein hal is Cl; X is —CH and R¹ is cyano.

13. The carbonylation according to claim 1, wherein hal is Br; X is —C—CH₃ and R¹ is cyano.

14. The carbonylation according to claim 1, wherein hal is Br; X is N and R¹ is methoxy.

15. The carbonylation according to claim 1, further comprising the step of reacting the compound of formula I with a compound of formula V to a compound of formula VI: 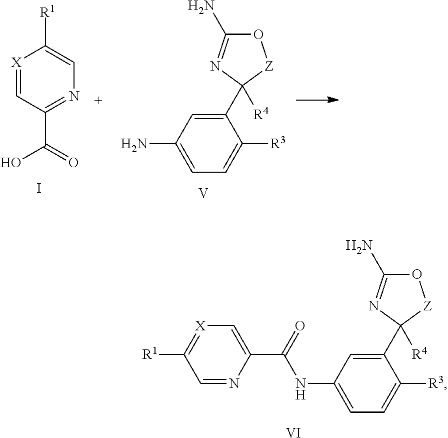
wherein R¹ and X have the meaning as described in any of claims 1-14, and
Z is —C(R⁵,R⁶)—C(R⁷,R⁸)—;
R³ is selected from the group consisting of
i) hydrogen, and
ii) halogen,
R⁴ is selected from the group consisting of
i) hydrogen,
ii) halo-C₁₋₆alkyl, and
iii) halogen,
R⁵, R⁶, R⁷ and R⁸ are each independently selected from the group consisting of
i) hydrogen,
ii) halo-C₁₋₆alkyl, and
iii) halogen;
or a pharmaceutically acceptable salt thereof.

16. The process according to claim 15, wherein R¹ is cyano.

17. The process according to claim 15, wherein R³ is F.

18. The process according to claim 15, wherein R⁴ is C₁₋₆alkyl.

19. The process according to claim 15, wherein R⁴ is methyl.

20. The process according to claim 15, wherein R⁵ and R⁶ are both hydrogen.

21. The process according to claim 15, wherein R⁵ and R⁶ are both halogen.

22. The process according to claim 21, wherein R⁵ and R⁶ are both fluoro.

23. The process according to claim 15, wherein R⁷ and R⁸ are both hydrogen.

24. The process according to claim 15, wherein R⁷ and R⁸ are both halogen.

25. The process according to claim 24, wherein R⁷ and R⁸ are both fluoro.

26. The process according to claim 15, wherein X is —CH.

27-28. (canceled)

29. A method for treating diseases and disorders characterized by elevated β-amyloid levels and/or β-amyloid oligomers and/or β-amyloid plaques and further deposits, comprising the step of administering to a patient in need thereof a therapeutically effective amount of a compound of formula VI according to claim 15.

30. A pharmaceutical composition comprising a therapeutically effective amount of a compound of formula VI according to claim 15 and a pharmaceutically acceptable carrier and/or a pharmaceutically acceptable auxiliary substance.

31. (canceled)

32. The method according to claim 29, wherein said disease is Alzheimer's disease.
